(19) United States
(12) Patent Application Publication
(10) Pub. No.: US 2013/0178733 A1
(43) Pub. Date: Jul. 11, 2013

(54) FUNCTIONAL BRAIN IMAGING FOR DETECTING AND ASSESSING DECEPTION AND CONCEALED RECOGNITION, AND COGNITIVE/EMOTIONAL RESPONSE TO INFORMATION

(71) Applicant: Daniel D. Langleben, Philadelphia, PA (US)

(72) Inventor: Daniel D. Langleben, Philadelphia, PA (US)

(21) Appl. No.: 13/624,681

(22) Filed: Sep. 21, 2012

Related U.S. Application Data
(63) Continuation of application No. 10/480,100, filed on Dec. 5, 2003.

(51) Int. Cl. A61B 5/055; A61B 5/145; A61B 5/16

(52) U.S. Cl. CPC A61B 5/055; A61B 5/164; A61B 5/14542
USPC 600/419; 600/410

(57) ABSTRACT

This invention provides method and system for measuring changes in the brain activity of an individual by functional brain imaging methods for investigative purposes, e.g., detecting and assessing whether an individual is being truthful or deceptive, and/or whether an individual has a prior knowledge of a certain face or object. The invention combines recent progress in medical brain imaging, computing and neuroscience to produce an accurate and objective method of detection of deception and concealed prior knowledge based on an automated analysis of the direct measurements of brain activity. Applying the paradigm developed from the deception model, and applying it to an individual viewing media information (e.g., audiovisual messages or movies, or announcements), the data is used to interpret the effect of the information on that individual. This permits the effective manipulation of the content of the media segments to achieve maximal desired impact in target populations or on specific individuals.

### REFERENCE TO RELATED APPLICATIONS

This application claims the benefit of and priority to now-expired U.S. provisional patent application Ser. No. 60/298,780, filed Jun. 15, 2001, and commonly owned, co-pending U.S. non-provisional patent application Ser. No. 10/480,100, the contents of each of which is herein incorporated in its entirety for any and all purposes.

### FIELD OF THE INVENTION

This invention relates generally to the field of utilizing measured changes in the brain activity of an individual by functional brain imaging methods for investigative purposes, e.g., detecting and assessing whether an individual is being truthful or deceptive, whether an individual has a prior knowledge of a certain face or object, as well as determining the cognitive/emotional response of an individual to media messages.

### BACKGROUND OF THE INVENTION

Recent progress in medical brain imaging, computing and neuroscience allows the creation of an accurate and objective method based on automated analysis of the measurements of brain activity by functional brain imaging for identification of cognitive activities of particular practical importance, namely 1) detection of deception and concealed prior knowledge and 2) assessment of the impact of the audiovisual media on target audiences.

Deception has major legal, political and business implications. Thus, there is a strong general interest in objective methods for determining with a high degree of certainty when one is intentionally lying (Holden, Science 291: 967 (2001)). According to the traditional approach, deception of another individual is the intentional negation of subjective truth (Eck, In Lies and Truth, McMillan, New York (1970)). This concept suggests that alteration of truthful response is a prerequisite of intentional deception.

Multichannel physiological recording (polygraph) is currently the most widely used technology for the detection of deception. The polygraph examination relies on the peripheral manifestations of anxiety (skin conductance, heart rate, and respiration), which deception is expected to induce (Office of Technology Assessment, 1983). The accuracy of this technique is limited by the variability of the association between deception and anxiety across individuals and within the same individual at different points in time (Steinbrook, N).

Scalp-recorded event-related potentials (ERPs) have also been used experimentally to detect deception. The P-300 (P-3) wave of the ERP appears in response to rare, meaningful stimuli with a 300- to 1000-ms latency (Rosenfeld, In Handbook of Polygraphy (Kleiner, ed.), pp. 265-286, Academic Press, New York, 2001). These series of voltage oscillations, which reflect the neuronal activity associated with a sensory, motor, or cognitive event, provide high temporal resolution, but their source in the brain cannot be uniquely localized (Hillyard et al., Proc. Natl. Acad. Sci. USA 95: 781-787 (1998)). As a result, ERP reflect cortical activity with a high temporal, but poor spatial resolution. Although amplitude and latency of the P-300 wave of the ERP have been associated with deception in the lab, this finding has not been successfully translated into a reliable lie-detection technology Rosenfeld, 2001). Thus, a need remains in the art for the development of a consistent, reputable and effective method and system for detecting deception in an individual by objective, rather than subjective means. Since deception-induced mood and somatic states appear to vary across individuals, a search for a marker of deception independent of anxiety or guilt is justified.

Medical Brain Imaging: All brain-imaging devices use energy to probe the area of interest and create a digital image that can be displayed graphically and manipulated statistically. In Magnetic Resonance Imaging SRI) the type of energy used to construct images is radio-frequency electromagnetic wave. The focus of medical brain imaging is either brain structure or brain function. Structural imaging emphasizes high spatial resolution and is used to detect stable anatomical changes in the brain, such as those occurring after strokes or degenerative diseases of the brain (e.g., Alzheimer's disease). The high spatial resolution is achieved at the expense of temporal (time) resolution, i.e., the detection of rapid brain changes during cognitive or other activity is not possible with structural imaging.

Both functional and structural imaging yields digital 2 or 3-dimensional maps of the brain that reflect tissue density (gray matter, white matter, fluid, tumor, etc.) or a measure of brain activity (e.g., rate of blood flow or metabolism). Functional brain imaging is performed with the same imaging equipment as structural imaging, to detect reversible changes in the brain that occur during cognitive, motor or sensory activity, such as finger tapping, remembering or deceiving. This requires a rate of acquisition of individual brain images in the order of magnitude of seconds (whole brain) or tens of milliseconds (single brain slice) that is much faster than is possible using structural imaging.

Functional magnetic resonance imaging (fMRI) comprises a group of MRI methods characterized by rapid acquisition of radiofrequency signals reflecting one of the parameters of regional neuronal activity in the brain, such as increased regional cerebral blood flow (rCBF) or change in the proportion of oxygenated hemoglobin associated with increased metabolic activity of a group of brain cells performing a certain motor, sensory or cognitive activity. The advantage that fMRI offers over EEG is that it can localize the source of changed signal with a spatial resolution in the order of 3 mm, while the source of signal in EEG can not be established with certainty.

Blood Oxygenation Level Dependent (BOLD) MRI is a variant of fMRI that is sensitive to the change in the ratio between oxygenated to deoxygenated hemoglobin (Oxy/Deoxy Hgb) in the small blood vessels supplying clusters of brain neurons. However, BOLD fMRI measures only the change in Oxy/Deoxy Hgb ratio, but not the absolute rCBF itself. This feature of BOLD fMRI demands that a baseline condition to which the brain activity during the condition of interest is to be compared, must be included in every BOLD fMRI experiment. This ratio is closely coupled to the neuronal rate of metabolism, which is in turn highly correlated with neuronal activity (Chen 1999). Thus, the change in Oxy/Deoxy Hgb is an indicator of neural activity in the brain.

Currently BOLD is the most commonly used fMRI technique, however other fMRI techniques, such as Arterial Spin Echo Labeling (ASL) fMRI may be used interchangeably with BOLD (Aguirre et al., Neuroimage 15: in press (2002)). In other fMRI techniques, absolute measures of the rCBF can be obtained.

Recent advances in computing speed and storage permit acquisition of an image of a single 4-mm slice of the brain in less than 100 mseconds. Twenty 4-mm slices cover most of the brain cortex, permitting acquisition of a whole brain image every 2 seconds. The pattern of the change in the Oxy/Deoxy Hgb is similar across a variety of cognitive and sensory tasks and is called Hemodynamic Response Function (HRF). Acquiring whole brain images every few (1-6) seconds allows monitoring and mapping of the HRF response to single stimuli during cognitive processes.

Unlike the ERP, the spatial resolution of functional magnetic resonance imaging (fMRI) exceeds that of any other brain imaging technique, while the temporal resolution is sufficient to resolve rCBF or Oxy/Deoxy Hgb changes occurring in response to either groups (blocks) or single cognitive events (e.g., a response to a question flashed on a screen). (Chen et al., In Functional MR, B. P. Moonen and Bandettini, eds., pp. 103-114, Springer-Verlag, New York, 1999).

The frequency and order of the stimuli which comprise an event-related MRI task affects the statistical power of the test. Until recently, the frequency of the brain hemodynamic response function (HRF, 1 cycle per approximately 15 seconds) limited the rate of stimuli presentation to 1 per 15 seconds. Recent work demonstrated a Fourier transform-based method to deconvolve the HRF response to individual stimuli that are presented at rates faster than the HRF frequency, if the inter-stimulus interval is variable. Such paradigms are termed “fast jittered event-related fMRI” (Burock et al., NeuroReport 9: 3735-3739 (1998)). This approach permits an order of magnitude increase in the number of stimuli presented per unit time, thus increasing the statistical power. Paradigms that are effective at a 1 per 15-second stimulus presentation rate can be converted into a fast jittered event-related fMRI paradigm to maximize the statistical power by these techniques.

Functional MRI imaging yields 2-dimensional maps of “raw” MRI signal, which are meaningless unless subtracted from the baseline or comparison condition (Friston et al., 1995a, 1995b). For example, in studying a response to light, activity in the occipital cortex during light is subtracted from activity in that region during darkness. The resolution of the system determines the dimensions of the smallest 3-D imaging unit, which is determined a “voxel” and is usually a 3 to 4-mm cube. The key steps in fMRI image analysis include motion correction, 3-D reconstruction of the 2-D data, “morphing” of the brain image of each individual to a standard template using a mapping coordinate system (Talairach et al., 1998). The resulting statistical image allows unique localization, and then comparisons between baseline and target conditions within and across subjects. The comparisons are voxel-by-voxel subtractions of the MRI signal in any two conditions (e.g., activity while seeing a familiar vs. unfamiliar face) made throughout the entire brain. The significance of the differences is determined using familiar two tailed t-tests, ANOVA or MANOVA, depending on the presence of additional non-imaging covariates of interest, such as polygraphic variables, gender, left-or-right-handedness, or—in this application—native language. The area commonly included in the analysis is often in the order of magnitude of 20-30,000 voxels, which requires a correction for multiple comparisons. The end result of this process is usually a map of above-threshold differences between two conditions expressed as t or F values.

Additional development in fMRI-research of higher cognitive functions is the ability of fMRI to distinguish brain activity pattern in response to a familiar vs. novel face or object (Opitz et al., Cereb. Cortex 9: 379-391 (1999); Senior et al., Cognitive Brain Research 10: 133-144 (2000); Wiser et al., J. Cogn. Neurosci. 12: 255-266 (2000)). Studies indicate that this effect takes place even in the absence of awareness (Milner, Philos. Trans. R. Soc. Lond. B. Biol. Sci. 352(1358): 1249-1256 (1997); Berns et al., Science 276: 1272-1275 (1997)). Moreover, different parts of the brain are activated in response to exposure to audiovisual stimuli (e.g., media) of different semantic categories, e.g., faces vs. furniture (Ishai et al., J. Cogn. Neurosci. 12: 35-51 (2000); Haxby et al., Science 293: 2425-2430 (2001); Haxby et al., Biol. Psychiatry 51: 59-67 (2002)).

Assessment of the impact of audiovisual media on target populations is of interest to the producers of such media (advertisers, filmmakers). Presently, such assessments are usually made by large scale and costly surveys of the subjective impressions of the target populations by following viewership (Nielsen's ratings) and also empirically. Such techniques are costly and limited in their ability to predict response. Moreover, they do not allow objective testing prior to the completion of the media segment by the time assessment that would permit adjustments in the content and form during production. Recently, the first attempt to use EEG/ERP to gauge brain response to media was made by Rossiter, J. Advertising Res. 41 (March-April 2001)). However, the limitations of the method described above for detection of deception with EEG limits the utility of this approach to the assessment of the media impact. As a result, there has been a need in the art for a reliable, yet simple and non-invasive method or system for predicting the impact of media messages on the public or sectors of the public.

The Guilty Knowledge Test (GKT): GKT is a method of polygraph interrogation that facilitates psychophysiological detection of prior knowledge of crime details that would be known only to a suspect involved in the crime (Lykken et al., Integr. Physiol. Behay. Sci. 26: 214-222 (1991); Elaad et al., J. Appl. Psychol. 77: 757-767 (1992)). The GKT has been adapted to model deception in psychophysiological (Furedy et al., Psychophysiology 28: 163-171 (1991); Furedy et al, Int. J. Psychophysical. 18: 13-22 (1994); Elaad et al., Psychophysiology 34: 587-596 (1997)) and ERP research (Rosenfeld et al., Int. J. Neurosci. 42: 157-161 (1988); Farwell et al., Psychophysiology 28: 531-547 (1991); Allen et al., Psychophysiology 29: 504-522 (1992)). In a typical laboratory GKT, the subject is instructed to answer “No” in response to a series of questions or statements, the answer to some of which is known to be “Yes” to both the investigator and the participant; however, the participant may be unaware of investigator's knowledge. An important distinction between the forensic and the laboratory GKT is that in the latter, the deception is endorsed by the investigator (Furedy et al., 1991).

While still conforming to the traditional definition of deception, committing experimental deception may not be perceived by the subject as an immoral act and is less likely to invoke guilt or anxiety than the forensic version. Consequently, a method that is sensitive to deception under experimental conditions is likely to be independent of anxiety and thus free of the limitations of the polygraph.

### SUMMARY OF THE INVENTION

It is an object of the present invention, particularly in light of recent terrorist activities against the United States, to provide a system and method or marker that permits the objective detection of deception by an individual; thus, permitting the reliable detection of criminal intent and conspiracies before innocent parties are harmed by the deception. Information about individuals or networks of individuals conspiring to commit acts of terror or drug trafficking is the single most important factor in protecting society by combating and preventing their activities. The principles of democracy limit the means available to law enforcement agencies for the interrogation of suspects and their collaborators, while intentional deception reduces the value and reliability of any information that is obtained.

Presently, polygraph is the only objective interrogative device in common use. But, as previously indicated, the validity and accuracy of polygraph results has been questioned because the polygraph monitors only the peripheral manifestations of the nervous system. However, the human brain, not the peripheral nervous system, is the ultimate location of the information sought by investigators. Moreover, variability in polygraph results can also arise from the association of emotional arousal (guilt or anxiety) with deliberate lying. False-positive results are common in anxious subjects in the setting of screening large numbers of largely innocent individuals, such as those taking place in relation to the anthrax attacks investigation. False negative results are especially likely with suspects trained in polygraph countermeasure techniques, and those with abnormal anxiety response to stress. Individuals with antisocial personality disorder, which is common in career criminals, may have reduced level of anxiety response to a variety of stimuli, including interrogation.

Thus, it is a primary object of the present invention to provide a general lie detection system and method based on an automated or semi-automated analysis of brain activity data acquired with direct imaging and mapping of individual brain activities by fMRI or other methods of measurement of brain blood flow and oxygenation.

It is also an object of the present invention to provide a method and system that apply the principles set forth in the fMRI deception paradigm to deception regarding acquaintanceship, e.g., to facial recognition. Specifically, this system and method will determine whether an individual is telling the truth or lying, and whether the subject is previously acquainted with another individual or is familiar with a particular object.

The test study presented in Example 1, provides a paradigm which is then subject to modification, and for which normative values are generated to establish the effects of relevant types of human variability (e.g., gender, mother-tongue language, handedness, and the like) on the brain response patterns established in the presented study. The thus-provided prototype is useful for the testing of “real life” suspects. Results of the prototype testing indicate that (a) cognitive differences between deception and truth have neural correlates detectable in an individual fMRI; (b) alteration of a truthful response is a basic component of intentional deception; (c) the anterior cingulate and the prefrontal cortices of the brain are components of the basic neural circuitry activated during deception in humans; and (d) MRI is a promising and effective tool in the study of deception and other cognitive process, relevant to lie detection, such as recognition of previously seen objects, which offers a significant new tool to the defense and criminal justice system and for use in many other areas in which detecting deception is of value.

The test study presented in Example 3, provides a paradigm which is then subject to modification, and for which normative values are generated to establish the effects of relevant types of individual variability (e.g., gender, socioeconomic status, age and the like) on the brain response patterns established in the presented study. The thus-provided prototype is useful for the testing of actual media segments. Results of the prototype testing indicate that (a) cognitive differences between two media segments of different semantic and emotional relevance have neural correlates detectable by fMRI; (b) MRI signal is correlated with subjective emotions induced by a media segment; and (c) MRI is a promising and effective tool in the study of group and individual response to media and in the manipulation of media content and form to achieve optimal desired and minimize the undesired response and impact.

Additional objects, advantages and novel features of the invention will be set forth in part in the description, examples and figures which follow, and in part will become apparent to those skilled in the art on examination of the following, or may be learned by practice of the invention.

### DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown.

FIG. 1 depicts a segment from the computerized GKT adapted for event-related fMRI. Each “Truth” (2 of Hearts), “Lie” (5 of Clubs), and “Control” (10 of Spades) was presented 16 times, each Non-Target card was presented twice. Stimulus presentation time was 3 seconds, inter-stimulus interval was 12 seconds, total number of presentations was 88. Order of presentation was pseudorandom (randomly predetermined).

FIG. 2 depicts a SPM {t} map projected over standard MRI template demonstrating significant increase in fMRI signal after “Lie” is compared with “Truth” in the ACC, the medial right SFG, the border of the left prefrontal cortex, the left dorsal premotor cortex, and the left anterior parietal cortex. Threshold of p was less than 0.01; corrected for spacial extent at p<0.05.

FIG. 3 depicts the average of statistically significant rCBF differences in 3 opiate-dependent patients when viewing a video containing heroin-related segments vs. neutral media segments, as demonstrated with ASL fMRI.

FIG. 4 depicts a high level of positive correlation between the reported subjective emotion of craving to use a drug and the strength of the MRI signal in the midbrain of patients addicted to the drug.

### DESCRIPTION OF PREFERRED EMBODIMENTS OF THE INVENTION

Deception, specifically “intentional deception,” is an act intended to create in the mind of the individual being deceived, a perception of reality which is different from the individual causing the deception, and in fact, usually different from objective reality. This invention provides a system and method by which regional brain activity in the deceiving individual, as elicited by that individual's inhibition of the truth response, comprises a marker for intentional deception. The invention is recognizes at least the following: (1) the difference in brain activity in an individual who is lying, and the same individual telling the truth can be detected and localized with fMRI; and (2) in normal adult human beings, a paradigm modeling deception, such as the GKT, activates parts of the cingulate and prefrontal cortex associated with altering the truth response into the deceptive response.

Although a detailed disclosure of the test study used to form the paradigm is presented by Example 1, a brief overview follows. A task was prepared that offers a formal, multiple choice type method of questioning an individual, wherein deception is modeled as intentional denial of the facts the individual believes to be true. For example if applied to a crime suspect, knowledge of the facts, and hence deceptions relating to those facts, indicates direct or indirect involvement (including witnessing) the crime. Results were generated using an event-related GKT and BOLD fMRI on a 4-Tesla (4-T) General Electric MRI scanner to compare MRI signals during deception and truthful responses in a representative sample of the population that performed the GKT. Data was analyzed automatically with statistical parametric mapping (SPM99).

Briefly, the approach is as follows. The rate and duration of stimulus presentation and the rate of acquisition of fMRI images of the brain (time of repetition (TR)) are synchronized via an electronic pulse emitted by the scanner at the start of each TR interval, which triggers presentation of the visual stimulus (e.g., photograph or a card) at a rate which is a multiple of the TR. There is thus a direct correspondence between individual stimuli and the fMRI images. Stimulus-dependent activation is assessed, for each individual voxel, via multiple regression of the time series of activation versus a set of lagged stimulus sequences, under the assumption that signal changes elicited by adjacent stimuli are linearly additive (Maccotta et al., 2001). This technique is termed “event-related fMRI” (Aguirre, In Functional MRI (Moonen and Bandettini, eds.), pp. 369-381, Springer-Verlag, New York, 1999). Mapping of the brain rCBF response to longer (20-30 seconds) trains (blocks) of closely spaced repeated stimuli is also possible and such paradigms are termed “block-design fMRI.”

MRI is the most established method for non-invasive imaging of brain activity, however additional experimental methods of measurement of regional cerebral blood flow and oxygenation, such as Near Infrared Spectroscopy (Villringer et al., Trends Neurosci. 20: 435-442 (1997)), which, once commercialized, could be used by an average practitioner in the present invention in the same fashion as fMRI. For example, the light source is coupled to the subject's head via fibre-optical bundles (optode). Since light is highly scattered after entering tissue a second optode, placed 2-7 cm away from the first can collect light after it has passed through the tissue beneath the optodes. The light-receiving optode is connected to a light detecting system such as a photomultiplier or a CCD camera. Several NIRS studies in recent years have demonstrated that changes in brain activity can be assessed non-invasively in adult human subjects. Several types of brain activity have been assessed, including motor activity, visual activation, auditory stimulation and performance of cognitive tasks. NIRS-parameters (oxy-Hb, deoxy-Hb, CO) exhibit typical responses to functional brain activation using a four-wavelength system. Most studies, until recently, were performed with single-site NIRS systems, but recently several studies have shown that multisite mapping of brain activity is also possible. Multi-optode arrays are now being developed that have been shown to permit functional neuroimaging. Nonetheless, fMRI is the technique of most relevance for the current purposes because it allows repeat studies of the same individual, is non-invasive (e.g., requires no IV lines or radiation exposure) and is a mature technology. The fMRI studies for the present invention utilized at high magnetic field scanner (4 T, rather than 1.5 T) because of the improved signal-to-noise ratio improvement over the conventional 1.5 T scanner (Maldjian et al., 1999). Alternative scanning mechanisms may be substituted therefor.

Standard approaches employing parametric statistics (Statistical Parametric Mapping or SPM99′) within the General Linear Model have already been developed and statistical packages for fMRI image analysis are commercially available. Statistical power analysis in MRI experiments is an area of intense investigation because its effects in cognitive MRI experiments are not well established, but it usually is in the 2-5% range.

The present invention is exemplified by a test version of the GKT, variations of which have been well validated as a model of deception, but have never before been combined with MRI measurements to detect the deception. Nor has any other type of deception model been previously combined with MRI to detect deception. However, when fMRI analysis was applied in the present invention, increased activity in the anterior part of the cingulate gyms (further named Anterior Cingulate Cortex or ACC), the right superior frontal gyms (SFG) and a contiguous area extending from the left lateral prefrontal to the left anterior parietal cortex (further named left lateral prefrontal cortex or the left PFC) were found to be specifically associated with deceptive responses. Thus, the results confirm that (a) cognitive differences between deception and truth have neural correlates detectable by fMRI imaging; and (b) ACC, SFG and PFC are components of the basic neural circuitry in an individual practicing deception. Additional regions indicative of deception include the left medial frontal gyri (MFG), the right MFG, the bilateral superior frontal gyri and the left orbital gyri.

The ACC and the dorsolateral prefrontal cortex (DLPFC) activation has been reported in executive function tasks involving inhibition of a “prepotent” (e.g., basic) response, divided attention, or novel and open-ended responses (Carter et al, Science 280: 747-749 (1998)). Recent fMRI studies manipulating the Stroop task, a response inhibition paradigm, have narrowed the role of the ACC to monitoring the conflicting response tendencies, and showed that the degree of right ACC activation is proportional to the degree of response conflict and inversely related to the left DLPFC activation (Carter et al., Proc. Natl. Acad. Sci. USA 97: 1944-1948 (2000); MacDonald et al., Science 288: 1835-1838 (2000)). Increased activation of the right ACC, during the “Lie” response indicates that a conflict with the prepotent response (Truth) and its' alteration are taking place.

Differential activation in the brain during the “Lie” also included the aspect of the right SFG (BA 8) contiguous with the ACC, suggesting functional continuity during the GKT deception (Kosli et al., Exp. Brain Res. 133: 5565 (2000)). Primate studies have demonstrated rich projections between the BA 8 and the ACC as well as the inhibitory role of BA 8 in previously learned forelimb movements (Oishi et al., Neurosci. Res. 8: 202-209 (1990); Bates et al., J. Comp. Neurol. 336: 211-228 (1993)). Consequently, increased activity at the junction of the left dorsal premotor and prefrontal cortices and the anterior parietal cortex may be related to increased demand for motor control directing right thumb to the appropriate response button during the “Lie” button press. This increase in activation appears to reflect additional effort needed to “overcome” the inhibited true response.

Importantly, the aforementioned brain regions were found to be more active during “tie” than “Truth,” but no brain regions were more active during “Truth” than “Lie.” This indicates that “Truth” is the baseline cognitive state and deception indeed requires performing a cognitive procedure on the truth which leads to extra brain activation during “Lie” but not “Truth,” as described above.

In the present invention the GKT was designed to minimize anxiety response, while maintaining the motivation to deceive with modest positive reinforcement (in this case by a small monetary reward). None of the participants reported any symptoms of subjective anxiety during or after the GKT scan. Similarly, the clinicians conducting the study found no activation of the regions frequently associated with positive skin conductance response, anxiety, or emotion (orbitofrontal cortex, lingual and fusiform gyms, cerebellum, insula, and amygdala) (Gur et al., J. Cereb. Blood Flow Metab. 7: 173-177 (1987); Chua et al., Neurollmage 9: 563-571 (1999); Critchley et al., J. Neurosci. 20: 3033-3040 (2000)). Thus, ACC activation does not appear to be a correlate of anxiety. Nevertheless, because parts of the ACC may be involved in emotional information processing, the present data alone can not definitively exclude anxiety or emotion-related activation (Whalen et al., Biol. Psychiatry 44: 1219-1228 (1998)).

Consequently, the present test study has certain recognized limitations stemming from paradigm design and the constraints imposed by the MRI environment, for which compensating considerations have been added.

First, under “field” conditions, deception involves elements of choice and more elements of risk and emotion than is the case in the test situation that follows. Recognizing that supplementing the GKT with a paradigm that allows the participant a choice in manipulating risk could reveal additional regions of deception-specific activation, such as the orbitofrontal cortex (Bechara et al., Cereb. Cortex 10: 295-307 (2000). Moreover, because a susceptibility artifact limits BOLD fMRI imaging of the orbitofrontal cortex, alternative imaging sequences offer certain advantages.

Second, the 12-second inter-trial interval of the event-related test design limited the number of stimuli that could be presented in a single session, and thus the statistical power of the findings. Consequently, the repetition of the Lie and Truth stimuli was necessary to amplify the inherently low power of event-related BOLD fMRI paradigms (Aguirre, 1999). However, even using a polygraph, Elaad reported no decline in the accuracy of detection of deception with repetitive GKT stimuli (Elaad et al., 1997). The present test GKT was controlled for both habituation and the “oddball” effect by equal repetition of all stimuli included in the analysis (Control, Lie, Truth). A modified event-related paradigm with faster stimuli presentation rate and variable inter-trial interval (“jitter”) could allow an even greater reduction in repetition of salient stimuli (Burock et al., 1998).

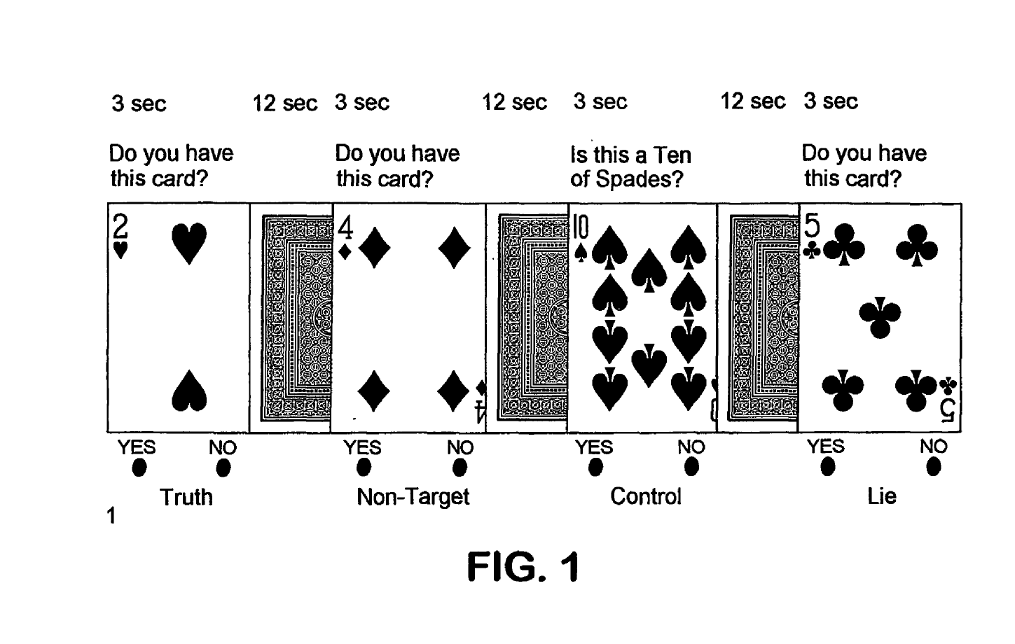

Third, the Truth and Lie cards (FIG. 1) differed in both suit and number. Shape and color discrimination have been associated with parietal and occipital, but not cingulate activation, making the graphic differences between the Truth and the Lie cards unlikely causes of ACC activation (Farah et al., Trends Cognit. Sci. 3: 179-186 (1999)). A proposal to resolve this question involves replication of the present findings with a GKT using playing cards that differ in number only, or that are simple number cards.

Finally, the present MRI data have not been correlated with ERP or polygraph recordings because of the limited reliability of polygraphy (Office of Technology Assessment, 1990). Simultaneous ERP and MRI recording is hampered by the strong magnetic field and is a focus of current research (Goldman et al., Clin. Neurophysiol. 111: 1974-1980 (2000)).

Although the system and method of the present invention are set forth in detail in the Examples, many of the variables may be substituted or altered so long as the changes are in keeping with the general principles defining the claimed invention. For instance, images of suspected collaborators or physical evidence can be substituted for the cards used in the Example. Other computer or scanner models or brands may be substituted if they perform similar functions to those that were used in the Examples. Such changes and substitution would be within the capability of the average clinician or practitioner of such assays and within the scope of the present invention.

In addition to detecting deception or concealed knowledge in defense and law enforcement, the applications of the present technology include civil law, commerce psychiatry and psychology. For example, it can be used for:

1) asserting innocence in civil, as well as criminal investigations (e.g. screening of thousands of federal employees in relation to the anthrax attacks investigations);

2) medicolegal applications, such as evaluating claims for psychiatric and other medical disability against private and government insurers; or

3) psychiatric diagnosis and objective assessment of the progress of psychotherapy as evidenced by an increase in brain activity characteristic of intentional denial instead of unconscious suppression, which is unlikely to produce deception-type brain response and assessment for false vs. true “recovered” memories (Schacter et al., Neuron 17: 267-274 (1996).

### EXAMPLES

The invention is further described by example. The examples, however, are provided for purposes of illustration to those skilled in the art, and are not intended to be limiting. Moreover, the examples are not to be construed as limiting the scope of the appended claims. Thus, the invention should in no way be construed as being limited to the following examples, but rather, should be construed to encompass any and all variations which become evident as a result of the teaching provided herein.

### Example 1

### A GKT Test Study

Twenty-three (23) healthy right-handed participants (11 men and 12 women) ages 22 to 50 years (average 32), education 12-20 years (average 16), were recruited from the University of Pennsylvania community. Participants were screened with Symptom Checklist-90—Revised (SCL-90-R) and a DSM-IV-based interview (American Psychiatric Association Diagnostic and Statistical Manual, 4.sup.th Edition (DSM-IV))-based interview to assure psychological normalcy before the scan. They were also questioned about symptoms of anxiety, if any, experienced during and/or after the scan {SCL-90-R items 2, 4, 12, 17, 23, 31, 39, 55, 57, 72, 78} (see survey published by Derogatis, et al., Br. J. Psychiatry 128: 280-289 (1976)).

A “high-motivation” version of the GKT described by Furedy et al., 1991, was adapted as follows: (1) instead of handmade cards with written numbers, numbered playing cards (FIG. 1) were used, (2) two non-salient card types were added to ensure alertness and attention and to control for the effect of repetition of the salient cards. The need for the multiple repetition of the salient stimuli and thus a special effort to maintain participants' alertness was dictated by the event-related fMRI paradigm design (Aguirre, 1999). Four (4) categories of cards were used: 5 of Clubs (“Lie”), 11 different numbered playing cards (“Non-Target”), 2 of Hearts (“Truth”), and 10 of Spades (“Control”).

The Lie, Non-Target, and Truth cards carried the question: “Do you have this card?” The Control was accompanied by a question “Is this the 10 of Spades?” to detect indiscriminate “No” responses. The Control forced the participants to read the questions on top of all cards, rather than give an indiscriminate “No” response. The Non-Target introduced an appearance of randomness and reduced habituation and boredom that is expected if only three cards were repeatedly presented over 22 minutes. Truth was presented the same number of times as Lie to control for the effect of repetition (habituation).

Participants were told that if they lied about any card other than the one hidden in their pocket the reward would be forfeited. This amounted to endorsing the truth about not having the Non-Target and Truth cards, denying the truth (lying) about not having the Lie card, and endorsing the truth about the Control being the 10 of Spades. Lie, Truth, and Control were presented 16 times, and each Non-Target was presented only twice, for a total of 88 stimuli. A random numbers generator was used to order the stimuli, which were presented for 3 seconds each. The inter-stimulus interval was 12 seconds (Aguirre, 1999), and thus the entire session lasted 1320 seconds (22 minutes).

PowerLab software (Chute et al., Behay. Res. Methods Instruments Comput. 28: 311-314 (1996) (MacLaboratory, Inc., Devon, Pa.) was used to assemble the GKT from scanned images of selected numbered playing cards and add-on graphics (FIG. 1).

All participants were familiar with card games, but had no history of problem gambling. Participants were asked to pick one of three sealed envelopes, all of which contained a $20 bill and a 5 of Clubs playing card. Participants did not know that all envelopes held the same contents. Participants were asked to secretly open the envelope, memorize the card, put it back in the envelope, and hide it in their pocket. Participants were told that they would be able to keep the $20 if they succeeded in concealing the identity of their card from a “computer” that would administer the GKT and analyze their brain activity during the MRI session. Participants were then positioned in a high field MR scanner (4 Tesla MRI scanner, GE Signa), equipped for echo-planar imaging.

A computer (Apple) running PowerLab and interfaced with a video projector was used to back-project the GKT onto a screen at the participants' feet, visible through a mirror inside the radiofrequency head coil. “Yes” or “No” responses were made with a right-thumb press on a two-button fiber-optic response pad (Current Designs, Philadelphia, Pa.). Responses were fed back to the Apple computer and recorded by the PowerLab. Image acquisition was synchronized with stimuli presentation in an event-related fashion. Sagittal T1-weighted localizer and a T1-weighted acquisition of the entire brain were performed in the axial plane (24 cm FOV, 256.times.256 matrix, 3-mm slice thickness). This sequence was used both for anatomic overlays of the functional data and spatial normalization of the data sets to a standard atlas.

Functional imaging was performed in the axial plane using multislice gradient-echo echo-planar imaging (21 slices, 5 mm thickness, no skip, TR 5 3000, TE 5 40, and effective voxel resolution of 3.75.times.3.75 3 4 mm. The fMRI raw echo amplitudes were saved and transferred to a memory source (Sun Ultrasparc 10, Sun Microsystems, Mountain View, Calif.) for offline reconstruction. Correction for image distortion and alternate k-space line errors on each image was based on the data acquired during phase-encoded reference imaging (Alsop, Radiology 197: 388 (1995).

Statistical analysis was performed as described by Friston et al., (Hum. Brain Mapping 2:

165-189 (1995a); Hum. Brain Mapping 2: 189-210 (1995b) using SPM99 (Wellcome Department of Cognitive Neurology, UK) implemented in Matlab (The Mathworks, Inc., Sherborn, Mass.), with an Interactive Data Language (IDL) (Research Systems, Inc., Boulder, Colo.) interface developed in-house. The T1-weighted images were normalized to a standard atlas (Talairach et al., In Co-planar Sterotaxic Atlas of the Human Brain. 3-Dimensional Proportional System: An Approach to Cerebral Imaging, Thieme, N.Y., 1988) within SPM99. Slice-acquisition timing correction was performed on the functional data using sync interpolation. Functional data sets were then motion corrected within SPM99 using the first image as the reference. Functional data sets were normalized to Talairach space using image header information to determine the 16-parameter affine transform between the data sets and the T1-weighted images (Maldjian et al., J. Comput. Assisted Tomogr. 21: 910-912 (1997), in combination with the transform computed within SPM99 for the T1-weighted anatomic images in Talairach space. The normalized data sets were resampled to 4×4×4 mm within Talairach space using sync-interpolation. The data sets were smoothed using a 12×12×12-mm full width at half-maximum Gaussian smoothing kernel.

For the statistical parametric mapping (SPM) analysis, a canonical hemodynamic response function with time and dispersion derivatives was employed as a basis function, with proportional scaling of the image means. Temporal smoothing, detrending, and high pass filtering were performed as part of the SPM analysis. SPM projection maps (SPMs) were generated using the general linear model (GLM) within SPM99. Within-subject contrasts between GLM regression coefficients were generated within SPM99 for the main contrast: “Lie vs Truth.”

A second-level analysis was performed to generate group SPMs using a random-effects model within SPM99 with the individual contrast maps (Holmes et al., NeuroImage 7: S754 (1988). The resulting SPM{t} maps of distribution of the values of T was transformed to the unit normal distribution SPM{Z} Both Z and T are basic statistical values available from standard tables expressing the difference between the observed frequency of an event and that an event is expected to occur by chance in a given number of trials. The higher the value of Z and T, the less likely the event to occur at random. P is the probability of certain value of Z or T, and thresholded at a P of 0.01, corrected for spatial extent (P<0.05), using the theory of Gaussian fields as implemented in SPM99. Anatomic regions were automatically defined using a digital MRI atlas (Kikinis et al., IEEE Trans. Visualization Comput. Graph. 2: 2223-2241 (1996)), which had been previously normalized to the same SPM99 Talairach template for use with the present fMRI data. The resultant thresholded SPM was overlaid on a standard Ti template with MEDx (MEDx 3.3; Sensor Systems, Inc., Sterling, Va.) software.

Subjects were excluded from analysis if they made more than two errors responding to the Truth or Lie stimulus or more than three errors total on the GKT. Participants were also excluded from analysis if their individual Z maps contained nonanatomical curvilinear change in Z values, indicating a motion artifact (distortion of the image by subjects' motion during the scan) (Hajnal et al., Magn. Reson. Med. 31: 283-291 (1994)). In fact, during the analysis, four participants were excluded because of motion artifact, and one because of a 100% error rate on the GKT. The correct response rate was 97 to 100%. In a total of 88 trials, nine participants made no errors, four made one error, three made two errors, and two made three errors. None made more than two errors on the Lie, Truth, or Control cards. Therefore, the final number of participants included in the analysis was 18.

Montreal Neurological Institute coordinates (SPM99 output) were converted into stereotactic Talairach coordinates (referred to as {x;y;z}) using a nonlinear transform (Duncan et al., Science 289: 457460 (2000)) and anatomical and Brodmann areas (BA) determined from the Talairach atlas (Talairach et al., 1988). Within SPM99, a “contrast” between condition A and condition B returns only positive differences (an increase); to detect a decrease a reversed subtraction (B minus A) was performed.

### Results:

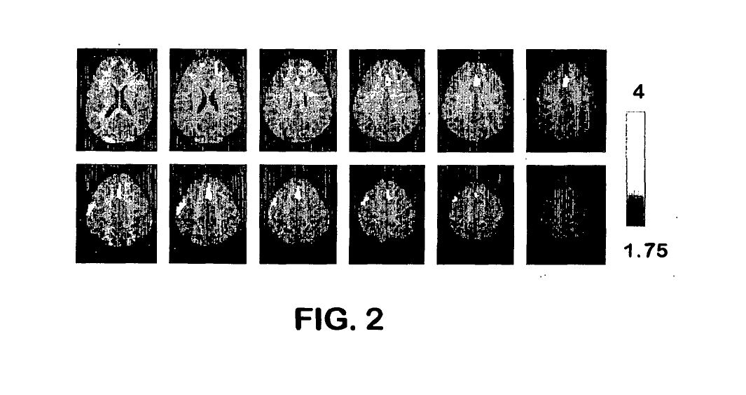

In the “Lie vs. Truth” contrast (Table 1, FIG. 2), there are two clusters of significant BOLD signal increase. The first is a 146-voxel cluster extending from the left anterior cingulate gyms (ACC) to the medial aspect of the right superior frontal gyms (SFG), including BA 24, 32, and 8, global activity peak at Talairach {x;y;z} coordinates {0;21;28} and local peaks at {4;33;43} and {0;26;47}. The second is a 91-voxel cluster, U-shaped along the craniocaudal axis, extending from the border of the prefrontal to the dorsal premotor cortex (BA 6, bordering on BA 3 and 4) and also involving the anterior parietal cortex from the central sulcus to the lower bank of the intraparietal sulcus (BA 1-3 to the edge of BA 40), with a global activity peak at {-63;-17;45} and local peaks at {-59;-10;41} and {-55;3;51}. There were no regions with significant signal decrease. See FIG. 2.

TABLE 1
Talairach coordinates, gyrus (Talairach et al., 1988)
and Brodmann Area (BA) locations of the peaks of activity
within clusters (FIG. 2) of significant fMRI signal differences
between “Lie” and “Truth” conditions.

| Cluster | | | | | | |
|---|---|---|---|---|---|---|
| size | Talairach coordinates | | | | | |
| (voxels) | Z | x | Y | z | BA | Gyrus |
| 146 | 3.8 | −1 | 16 | 29 | 24; 32 | Anterior cingulate |
| — | 3.17 | 3 | 28 | 43 | 6; 8 | Right superior frontal |
| | 3.15 | 0 | 24 | 52 | 8 | Superior frontal |
| 91 | 3.58 | −57 | −23 | 41 | 1; 2; | Left postcentral |
| | | | | | 3; 40 | |
| — | 3.40 | −54 | −15 | 38 | 3; 4; | Left pre- and |
| | | | | | 6 | postcentral |
| — | 3.19 | −50 | −3 | 49 | 6 | Left precentral |

Note.
Voxel level threshold T = 2.57, P < 0.001 uncorrected and 0.05 corrected for multiple comparisons, spatial extent threshold >80 voxels. Bold numbers correspond to a global peak of the cluster; italics represent local peaks within same contiguous cluster.

### Conclusions:

The results demonstrate that there are measurable difference between lying and telling the truth using event-related fMRI and the GKT model of deception. This finding indicates that there is a neurophysiological difference between deception and truth at the brain activation level that can be detected with fMRI. The anatomical distribution of deception-related activation indicates that deception involves conflict with, and alteration of, the prepotent (truthful) response. Further refinements of the paradigm design and image analysis methodology involving e.g., testing the effect of handedness, language or gender, or creating grades of deception based upon familiarity in the GKT, or testing the effect of implemented counter-measures by the subject (such as, nor responding to questions or commands in response to the presented stimuli) could further increase the salience and the statistical power of the simulated deception paradigms and establish an activation pattern predictive of deception on an individual level.

### Example 2

### Recognition of Familiar Faces

A conspiracy suspect trying to intentionally deceive an investigator about being acquainted with another individual (e.g., a co-conspirator) exhibits two parameters of brain function detectable by fMRI. The first is intentional denial of recognizing the co-conspirator (or his/her image). The second is response to a familiar face or object, which is different from the response to a novel face or object.

Studies of brain activity patterns during facial recognition have shown significant differences in the brain response to familiar vs. novel faces as well as the effect of the degree of prior familiarity with the displayed face (Haxby, 2002; Glahn et al., 1997; Henson et al., 2001; Schlack et al., 2001, Gobbini et al., 2001). Thus, when the principles of Example 1 are applied to the question of whether an individual recognizes a face or not, the present data indicates that when faces are used as stimuli in a GKT type paradigm a response is as strong or stronger (in amplitude and/or spatial distribution) than the GKT paradigm established with playing cards.

Studies indicate that this effect takes place even in the absence of awareness [Milner, 1997 #111; Berns et al. Science 276: 1272-1275 (1997). Ishai et al., J. Cogn. Neurosci. 12: 35-51 (2000); Haxby et al., Biol. Psychiatry 51: 59-67 (2002). Consequently, the principles set forth in the fMRI deception paradigm of Example 1 are applicable to deception regarding acquaintanceship and are combinable sequentially or serially with mapping the brain activity associated with novel vs. familiar facial or object recognition without deception.

### Example 3

### Brain Response to Media Information

The principles set forth in the fMRI deception paradigm of Example 1 may also be applied to individuals viewing media information, such as movies, video film clips, or advertising. Although in this case, rather than examining for deception, the data is used to interpret the effect of the information on the individual. This uses the known patterns of brain response, e.g., aversive, pleasurable, exciting or memory-evoking stimuli to adjust media content to achieve a desirable impact. This study explores the use of magnetic resonance signal as a marker of cognitive (e.g., attention) and emotional (e.g., arousal) responses to commercial audiovisual media Subjects are selected and analyzed as in Example 1 with certain modifications in the presentation and evaluation of the signals and resulting data.

### Data Acquisition:

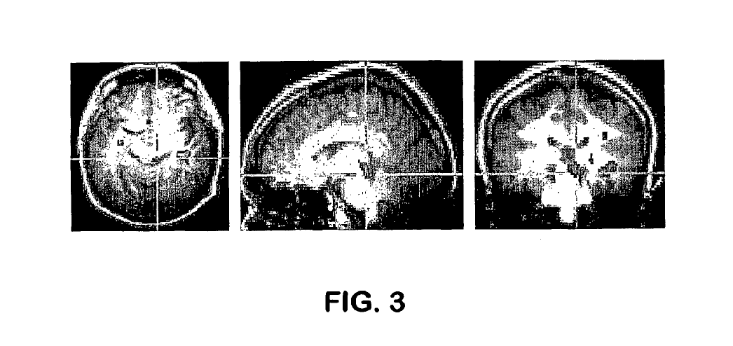

Subjects view the baseline media segment (control material) followed by the target media segment of same duration (While randomizing the order of the drug and neutral videos would remove the risk of systematic error due to MRI system drift, data acquired by the inventors indicates significant carry-over effects from the drug to the neutral cue). The target film used depicts two male heroin users engaged in drug-specific dialogue while: preparing and injecting simulated heroin. The baseline film is a nature film about the life of hummingbirds. FIG. 3 depicts an averaging of the rCBF differences between the brain response to a movie about heroin use and a movie about hummingbirds in 3 opiate-dependent patients as determined by with ASL fMRI projected over T1 MRI in Talairach space. Both films have been validated by correlation with skin conductance response and used in several previous studies at the inventor's laboratory.

Imaging consists of a sagittal scout scan (TRJTE=500/10 mseconds, 128.times.256, 5 mm thick, 2 minutes), an anatomical scan using 3D inversion recovery (IR) prepared spoiled GRASS (TR/TEM=33/7/400 mseconds, 192.times.256, 124 slices, 1.5 mm thick), followed by the fMRI using the arterial spin labeling (ASL) perfusion sequence (TR/TE=3400/18 mseconds, 64.times.40, 10 slices, 50 mseconds acquisition time/slice, 8 mm thick/2 mm sp, resolution 3.75.times.3.75.times.10 mm, FOV 24 cm, 180 repetitions, 10 mins). The ASL sequence consists of interleaved global (control) and slice-selective (label) inversion recovery gradient echo echoplanar acquisitions. A specific sharpedge pulse (FOCI) is applied for spin labeling to minimize the system error between acquisitions. The duration of the tagging bolus is defined by playing out a saturation pulse at the tagging region at 800 ms after the FOCI pulse, followed by a 1-second post-labeling delay before image acquisition. The total time in the scanner is about 30 minutes. Heart rate is obtained continuously and sampled every 30 seconds with a pulse oxymeter attached to subject's finger.

Assessment of the desire to use drugs depicted in the target segment and other subjective feelings, such as aversion, sexual arousal and remembering, are performed at fixed intervals or continuously throughout the session. Subjects use a response pad with multiple buttons, which permit them to communicate the degree to which they experience the above feelings to the investigator. Additional parameters such as skin conductance, penile tumescence, heart and respiratory rate and blood pressure are also collected as needed.

### Procedures:

After informed written consent, subjects are placed in the scanner. Video segments are projected onto a screen at the subject's feet and viewed with the aid of prism glasses attached to the inside of the radio frequency head coil. The sound is delivered by air conduction through plastic tubes threaded through earplugs that attenuate scanner noise. Videos are 10 minutes in duration, and are preceded and followed by a 4-minute blank gray screen during which VAS is administered and MRI is halted. VAS is used to index the change in cue-induced heroin craving. Subjects respond using a fibro-optic response pad.

TABLE 2
MRI session timeline indicating onset of the variables in
terms of time elapsed from beginning of the imaging session.

| | Elapsed time (min) | | | | |
|---|---|---|---|---|---|
| | 0 | 6 | 16 | 20 | 30 |
| Structural | x | | | | |
| MRI | | | | | |
| fMRI | | x | | x | |
| Target | | | | x | |
| segment | | | | | |
| Non-target | | x | | | |
| segment | | | | | |
| Subjective | x | x | x | x | x |
| symptoms | | | | | |

x indicates alternative (counterbalanced) order.

### Data Analysis:

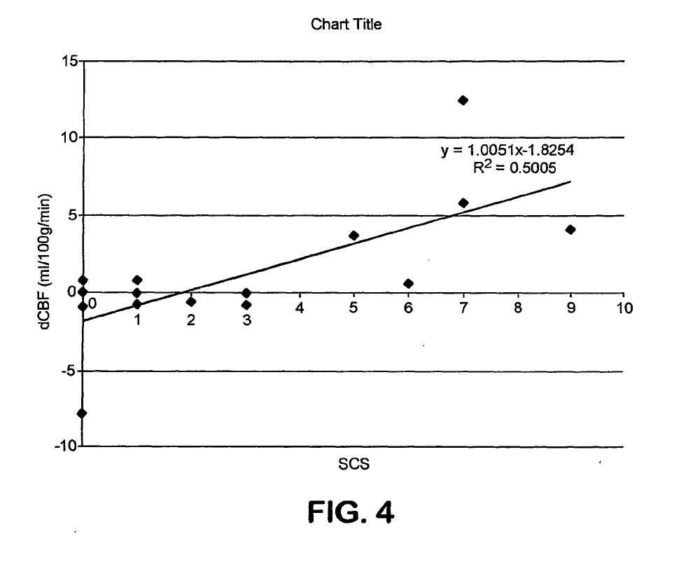

Data is reconstructed offline, corrected for motion artifacts and smoothed using SPM99′ (28, http://www.fil.ion.ucl.ac.uk/). The series of label images are shifted in time by one TR using linear or sync interpolation. Perfusion contrast images are generated by pairwise subtraction between the time-matched label and control images. FIG. 4 depicts the correlation between the change in the desire to use heroin and the change in rCBF in the midbrain area. Conversion to CBF values are effected using the general PASL perfusion model. CBF signals during the drug and non-drug video are compared within subjects using SPM99.

Individual activation maps (either beta or correlation coefficient) are normalized to Talairach space and correlated with methadone plasma levels and the heart rate to detect the brain areas associated with opiate craving and physiological parameters within both the patients and the controls. ANOVA analysis is performed on the normalized individual data to study the effects of drug cue and testing population, followed by region-of-interest analysis to further study the temporal evolvement of the time-course of the CBF change in these detected brain regions.

### Results:

1) Media segment of high emotional value for the target population elicits a different brain response than a media segment of neutral value in the midbrain, the thalamus, the insula and the amygdala. This effect was not observed in control subjects who were not addicted to heroin, nor in brain regions that were not involved in the mediation of the reward and motivation, such as the occipital cortex.

2) Brain response in some of these regions (midbrain) is correlated with the subjective emotions of the audience.

3) Perfusion fMRI at 4-T is a promising technique for the study of media impact on target populations, as well as individuals.

The method herein described is, therefore, useful for the effective manipulation of the content of the media segments to achieve maximal desired impact in target populations or on specific individuals.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

I claim:

1. A computer-implemented method to objectively determine if an individual is providing a deceptive response to an inquiry, comprising:
a. obtaining functional magnetic resonance image (fMRI) data of at least one cortical portion of the individual's brain in response to a test inquiry to acquire structural data reflecting at least one parameter of regional neuronal activity in the cortical portion(s) of the individual's brain, wherein the cortical portion of the individual's brain is selected from the group consisting of a left lateral prefrontal cortex, anterior cingulated cortex, orbitofrontal cortex, premotor cortex, parietal cortex, left medial frontal gyri, right medial frontal gyri, bilateral superior frontal gyri, and left orbital gyri, wherein the parameter(s) optionally is(are) increased cerebral blood flow and/or a change in proportion of oxygenated hemoglobin associated with increased metabolic activity of brain cells within such portion(s) of the individual's brain;
b. using a computer configured to process the structural data to determine a degree of cortical cognitive brain activity for the at least one cortical portion of the individual's brain as the individual formulates her/his response to the inquiry;
c. using a computer configured to compare the determined degree of cortical cognitive brain activity for the at least one cortical portion of the individual's brain to a predetermined degree of cortical cognitive brain activity for the same portion(s) of the individual's brain for a known truthful response to a previous inquiry to determine whether the response to the test inquiry is deceptive; and
d. using a computer configured to output whether the response to the test inquiry is deceptive to output whether the response is deceptive.

2. A method according to claim 1 wherein the data is T1-weighted data.

3. A method according to claim 1 that further comprises recording the response(s) made by the individual to the test inquiry(ies).

4. A method according to claim 3 further comprises recording the response(s) made by the individual to the previous inquiry(ies).

5. A method according to claim 4 that further comprises synchronizing in time the fMRI data and the response(s) made by the individual.

6. A method according to claim 1 further comprising using a computer to generate one or more fMRI-derived images of the at least one cortical portion of the individual's brain.

7. A method according to claim 6 that further comprises synchronizing in time the fMRI-derived images with recorded responses to test and/or previous inquiries.
